(19) United States
(12) Patent Application Publication
(10) Pub. No.: US 2022/0024879 A1
(43) Pub. Date: Jan. 27, 2022

(54) COMPOUNDS FOR TREATMENT OF HEPACI VIRUS INFECTION AND METHOD FOR DETERMINING THERAPY OF HEPACI VIRUS INFECTION, IN PARTICULAR, HCV INFECTION

(71) Applicants: TWINCORE ZENTRUM FÜR EXPERIMENTELLE UND KLINISCH INFECTIONSFORSCHUNG GMBH, Hannover (DE); GOTTFRIED WILHELM LEIBNIZ UNIVERSITÄT HANNOVER, Hannover (DE)

(72) Inventors: Thomas PIETSCHMANN, Hannover (DE); Dominic BANDA, Ghent (BE); Wladimir SOLODENKO, Hannover (DE)

(21) Appl. No.: 17/414,579

(22) PCT Filed: Dec. 17, 2019
(86) PCT No.: PCT/EP2019/085576

§ 371 Date: Jun. 16, 2021

(30) Foreign Application Priority Data
Dec. 17, 2018 (EP) 18212936.1

(51) Int. Cl. C07D 241/04; A61P 31/14

(52) U.S. Cl. CPC C07D 241/04; A61P 31/14

(57) ABSTRACT

In a first aspect, the present invention relates to new compounds based on diphenylpiperazine and diphenylpiperidine structures. In particular, the present invention provides new flunarizine derivatives having improved hepaci virus infection inhibitory activity. In a further aspect, the present invention relates to a pharmaceutical composition containing said compound as well as the use of said pharmaceutical composition and the compounds according to the present invention in preventing or treating hepaci virus infection, in particular, HCV virus infection, like HCV of genotype 2. Moreover, a method for determining effectiveness of prophylactic or therapeutic treatment of hepaci virus, like HCV infection as well as a method for determining the therapy regimen of an individual afflicted with hepaci vims infection including HCV infection is provided. Said method is based on determining the sequence or interfacial hydrophobicity of the hepaci virus E1 protein. This may include determining the presence of mutations at predetermined positions of the E1 sequence. Based on determining the interfacial hydrophobicity of the E1 protein, the sensitivity to a diphenylpiperazine or diphenylpiperidine based hepaci virus inhibitor as well as a phenothiazine and cycloheptenepiperidine based hepaci virus inhibitor can be determined. When the central hydrophobicity region is disrupted or the hydrophobicity is below zero applying the Wimley-White hydropathy plot, or the mutations at positions 290, 299, 301 and 310 of SEQ ID No. 1 are present, it is submitted that the sensitivity against said compounds is reduced. Hence, it is possible to determine the therapy regimen of an individual afflicted with hepaci virus infection or being a risk of being afflicted with hepaci vims infection, in particular HCV infection like HCV genotype 2 infection.

In a first aspect, the present invention relates to new compounds based on diphenylpiperazine and diphenylpiperidine structures. In particular, the present invention provides new flunarizine derivatives having improved hepaci virus infection inhibitory activity. In a further aspect, the present invention relates to a pharmaceutical composition containing said compound as well as the use of said pharmaceutical composition and the compounds according to the present invention in preventing or treating hepaci virus infection, in particular, HCV virus infection, like HCV of genotype 2. Moreover, a method for determining effectiveness of prophylactic or therapeutic treatment of hepaci virus, like HCV infection as well as a method for determining the therapy regimen of an individual afflicted with hepaci virus infection including HCV infection is provided. Said method is based on determining the sequence or interfacial hydrophobicity of the hepaci virus E1 protein. This may include determining the presence of mutations at predetermined positions of the E1 sequence. Based on determining the interfacial hydrophobicity of the E1 protein, the sensitivity to a diphenylpiperazine or diphenylpiperidine based hepaci virus inhibitor as well as a phenothiazine and cycloheptenepiperidine based hepaci virus inhibitor can be determined. When the central hydrophobicity region is disrupted or the hydrophobicity is below zero applying the Wimley-White hydropathy plot, or the mutations at positions 290, 299, 301 and 310 of SEQ ID No. 1 are present, it is submitted that the sensitivity against said compounds is reduced. Hence, it is possible to determine the therapy regimen of an individual afflicted with hepaci virus infection or being a risk of being afflicted with hepaci virus infection, in particular HCV infection like HCV genotype 2 infection.

### PRIOR ART

Hepatitis C virus (HCV) is a highly variable, enveloped virus of the family Flaviviridae. According to sequence analysis, viral isolates are classified into seven genotypes and 86 subtypes. HCV particles harbor a plus-strand RNA genome of positive polarity that encodes a polyprotein comprising structure proteins, the P7 ion channel and various non-structural proteins. Virus particles are composed of the core protein that encases the viral RNA, and of the envelope 1 protein (E1) and envelope 2 protein (E2). These are embedded in the viral lipid membrane. Virion-associated E1 and E2 glycoproteins coordinate interactions with cellular receptors, cell uptake and membrane interfusion which is triggered by the low pH in cellular endosomes.

Hepatitis C virus is a member of the hepaci virus genus. The Hepatitis C virus is also identified as the species hepaci virus C whereby humans are the only natural host. Additional hepaci virus are described, namely, hepaci virus A to N. Based on genetic differences between HCV isolates, the Hepatitis C virus species is classified into 6 genotypes (genotypes 1 to 7) with several subtypes within each genotype. Further differentiation in subtypes is done by separating these isolates in quasispecies. That is, a HCV of a genotype 2a is a HCV virus of genotype 2 with subtype a.

Chronic Hepatitis C virus infection is associated with severe liver diseases including hepatitis, cirrhosis and hepatocellular carcinoma. The WHO estimates that approximately 71 million individuals are chronically infected and at risk of developing disease. That is, HCV has a long persistence in humans without breakout of any symptoms of disease.

The licensing of multiple classes of directly acting antivirals (DAA) has in recent years revolutionized antiviral treatment. These drugs either target a viral NS3 protease, the NS5A phosphoprotein or the NS5B polymerase. Combination therapies involving these drugs are active against viruses from genotype 1 to 6 and reach curing rates of greater than 95%. Although 95% of cases may be cured, these treatments are costly and in some cases viral resistance can develop.

Resistance associated substitutions (RAS) have been observed for all classes of DAA and is associated with treatment failure. Although viral resistance and treatment failure occur infrequently, due to the relative short history of these new treatments, it is difficult to predict to which level salvage therapies including novel drug classes will be required in the future.

Cell entry inhibitors comprise part of HIV-1 combination therapies, and are in clinical development for treatment of Hepatitis B and Hepatitis delta virus infections. Also in case of HCV, cell entry inhibitors which either target viral components or cellular factors have emerged as interesting class of inhibitors, Colpitts C. C. et al., ACS infect Dis. 2017; 3:620-623. Preclinical studies have confirmed their efficacy in vitro and in vivo and have shown that combined with DAAs they prevent breakthrough of antiviral-resistant HCV. In the course of several independent screening campaigns involving various compound libraries including for instant the NRH clinical compound (NCC) collection or the library of pharmacologically active compounds (NOPAC 1280), a number of HCV entry inhibitors were discovered, see Pietschmann T., J Virol 2017; 91.

These HCV entry inhibitors have diverse but related chemical scaffolds including molecules from the group of diphenylpiperazines, diphenylpiperidines, phenothiazines, thioxantenes, and cycloheptenepiperidines. This group includes licensed drugs with neuroleptic or anti-histamine activity, like flunarizine and chlorcyclizine. That is, recently it has been reported that flunarizine as a clinically improved ion channel inhibitor and neuroleptic, inhibits HCV cell entry by preventing viral membrane fusion, see Perin, et al., 2016, Hepatology, 63, 49-62. It has been shown that fluphenazine, a phenothiazine, and pimozide, which both are structurally related to flunarizine, inhibit HCV infection by common mode of action. Further, it has been shown that these compounds specifically inhibit HCV entry and not infection by other enveloped viruses. Others reported that chlorcyclizine, a diphenylpiperazine and antihistamine that is structurally related to flunarizine, inhibits HCV infection, see He et al., Science Translation in Medicine 2015, 7, 282 RA 249. Antiviral activity in the nanomolar range and proven in vivo efficacy render some of these compounds attractive candidates for further development as HCV entry inhibitors. However, it remains unclear if these diverse compounds share their mode of action, if they target a common virus, and the susceptibility of virus against these inhibitors.

However, there is an ongoing need for new therapeutics for preventing and treating hepaci virus infection in a subject, in particular, for preventing and treating HCV infection overcoming known resistance of HCV inhibitors. Moreover, there is a need to address current and future problems associated with viral drug resistance to known drugs.

### BRIEF DESCRIPTION OF THE PRESENT INVENTION

The first aspect, the present invention relates to a compound of the general formula I

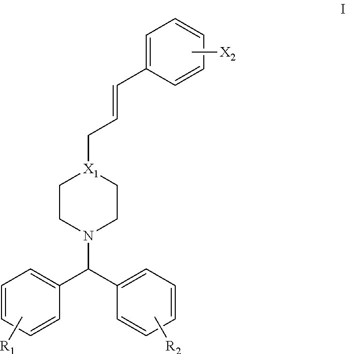

wherein R₁ and R₂ are independently from each other selected from a group of H, Halogen or a C₁-C₄ alkyl group whereby at least one of R₁ and R₂ is a halogen, R₁ and/or R₂ may be present once, twice, three, four or five times at the aryl group;

### X₁ is C or N;

X₂ is selected from a OR₃ group or a NR₄R₅ group, or is N₃ wherein
R₃ is H or a C₁-C₄ alkyl group, and wherein X₂ may be present once, twice, three, four or five times at the aryl group;
R₄ and R₅ are independently from each other selected from the group of H, N, C₁-C₄ alkyl group, and (CH₂)ₙ—CR₆R₇R₈;
wherein R₆ and R₇ are independently from each other selected from H or a C₁-C₄ alkyl group, and
R₈ is H, C₁-C₄ alkyl group or a cyclopropanyl group;
n is 0 or 1;
or salts, solvates or hydrates thereof.
It is preferred that X₂ is present once and/or is in para-position.
The present inventors recognized that the compounds of the general formula I have improved activity as hepaci virus membrane fusion inhibitor, in particular, as HCV inhibitor. The activity of said compounds according to the present invention is superior over the activity of flunarizine as well as chlorcyclizine described in the art.

In a further aspect, the present invention provides a pharmaceutical composition containing the compounds according to the present invention. Further, the compound according to the present invention as well as the pharmaceutical composition according to the present invention is for use in preventing or treating hepaci virus infection. Hepaci virus infection include non-primate hepaci virus like non-primate hepaci virus from horse, rat, gorilla, rodent, bat and bovine. In an embodiment, the hepaci virus infection is a hepatitis C virus infection in particular, HCV of genotype 2 (HCV GT2).

In another aspect, the present invention provides the compound according to the present invention or a pharmaceutical composition according to the present invention in combination with a further anti hepaci virus inhibitor known in the art, in particular, direct antiviral agents targeting viral NS3 protease NS5A phosprotein or NS5B polymerase.

Additionally, the present invention relates to a method for determining effectiveness of prophylactic or therapeutic treatment of hepaci virus infection, like HCV infection, in particular, HCV type 2 infection, comprising:
Determining the interfacial hydrophobicity of E1 of the hepaci virus, like E1 of the HCV strain, using the Wimley and White interfacial hydrophobicity scale;
Determining the sensitivity to a diphenylpiperazine or diphenylpiperidine based hepaci virus inhibitor as well as phenothiazine and cycloheptenepiperidine based hepaci virus inhibitors, in particular, a compound according the present invention,
   wherein a disrupted central hydrophobic region of the E1 protein or a hydrophobicity of the E1 protein lower than zero at the next following His residue, as has been determined with the Wimley-White hydropathy plot is indicative for reduced sensitivity to a diphenylpiperazine or diphenylpiperidine based hepaci virus inhibitor as well as phenothiazine and cycloheptenepiperidine based hepaci virus inhibitors.

In another aspect, the present invention relates to a method for determining the therapy regimen of an individual afflicted with hepaci virus infection or being at risk of being afflicted with hepaci virus infection, in particular, having or being at risk of HCV infection, comprising the step of
Providing a sample obtained from said individual and determining the interfacial hydrophobicity of the E1 protein of the hepaci virus, like E1 of the HCV or its homolog from hepaci virus, in particular, using the Wimley-White hydropathy plot;
Analyzing whether the central hydrophobicity region is disrupted or the hydrophobicity is below zero at the next following His residue, in particular, the hydrophobicity is below zero applying the Wimley-White hydrophathy plot;
Determining the therapy regimen wherein no inhibitor of the E1 protein of hepaci virus, like of HCV or its homolog, in particular, a diphenylpiperazine or diphenylpiperidine based HCV inhibitor as well as phenothiazine and cycloheptenepiperidine based hepaci virus inhibitors is applied when the central hydrophobicity region is disturbed or the hydrophobicity is below zero at the next following His residue applying the Wimley-White hydropathy plot.

### BRIEF DESCRIPTION OF THE DRAWINGS

FIG. 1. Resistance mutations to flunarizine confer cross-resistance to structurally related HCV entry inhibitors. Chemical scaffolds of recently described HCV entry inhibitors of the diphenylpiperazine, diphenylpiperidine, thioxanthene, phenothiazine, and cycloheptenepiperidine families, Curcumin and BJ486K, two HCV entry inhibitors.

FIG. 2. p-methoxy-flunarizine exhibits enhanced antiviral activity and improved therapeutic index. A library of new flunarizine derivatives was synthesized and screened for antiviral activity in a viral whole life cycle assay (Table 1). (A) Structure of flunarizine, of p-methoxy-flunarizine and summary of structure activity relationship of flunarizine-related molecules. Whole life cycle JcR2a infection assay in presence of increasing doses of flunarizine (B) and p-methoxy-flunarizine (C), respectively. Cells are transfected with JcR2a and compounds are added 4 h after transfection. HCV RNA replication is determined by quantifying renilla luciferase activity in transfected cells after 48 h. Cytotoxicity of compounds is quantified at the same time point using cellular fluc expression. Culture fluid of the transfected cells is passed on to naïve cells where renilla luciferase activity is determined 48 h later. (D) Comparison of antiviral activity of p-methoxy-flunarizine, chlorcyclizine and flunarizine as determined by the HCV whole life cycle infection assay (E) Antiviral activity of p-methoxy-flunarizine was examined by using given chimeric reporter viruses. Mean values of three biological replicates +/−SD are given.DMSO was used as vehicle control.

FIG. 3. Viral determinants within E1 control sensitivity to flunarizine. Susceptibility of chimeric JcR2a reporterviruses with J8-(GT2b) derived E1 regions. Infection is expressed relative to control infections in presence of solvent (DMSO). Mean values of three biological replicates +/−SD are given.

FIG. 4. Four conserved residues proximal to flunarizine resistance mutations and the putative fusion loop govern susceptibility to HCV membrane fusion inhibitors. (A) Sequence alignment of E1 protein region covering the putative fusion loop, flunarizine resistance mutations and region 3 of E1. Four GT2a-derived strains susceptible to flunarizine are given at the top, and four GT2b-derived isolates resistant to flunarizine are plotted below. Susceptibility of these strains to flunarizine was examined in Perin et al. Flunarizine resistance mutations are M267V and Q289H, fully conserved residues that distinguish sensitive and resistant strains are boxed. (B) Inhibition of JcR2a reporter viruses by given entry inhibitors. Infection is expressed relative to control infections in presence of solvent (DMSO). Mean values of three biological replicates +/−SD are given.

FIG. 5. Predicted hydrophaty of HCV E1 proteins with a focus on the central region encompassing the putative fusion loop and residues governing sensitivity to flunarizine. (A) Wimley-White hydropathy plot for the J6-E1 protein. Predicted hydrophobic regions are indicated by thick black bars on top of the smoothed predicted hydropathy (thin black line). The primary amino acid sequence of the central E1 region from residues 264 to 312 is provided above. The putative fusion loop is indicated by a bar (Aa 265-287), E1 region 3 section 1 (Aa 267-289) and section 2 (290-312) characterized in this study are indicated by bars. Residues conserved among flunarizine resistant HCV GT2b strains are plotted in bold face directly below the J6 sequence. Neutralized histidine residues are shown by circles. (B-G) Comparison of predicted hydropathy of central E1 region (residues 264-312) between J6-E1 and given alternative E1 proteins. Neutralized histidines are shown in circles given for J6 and for the alternative E1 proteins.

### DETAILED DESCRIPTION OF THE PRESENT INVENTION

The present invention refers to a compound of general formula I

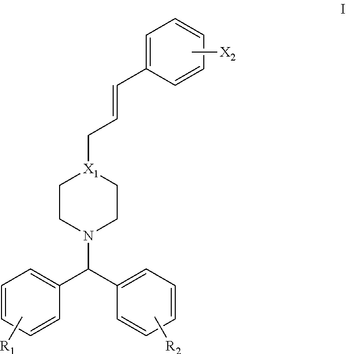

wherein R₁ and R₂ are independently from each other selected from a group of H, Halogen or a C₁-C₄ alkyl group whereby at least one of R₁ and R₂ is a halogen, and R₁ and/or R₂ may be present once, twice, three, four or five times at the aryl group;

### X₁ is C or N;

X₂ is selected from a OR₃ group or a NR₄R₅ group, or is N₃ wherein
R₃ is H or a C₁-C₄ alkyl group, and X₂ may be present once, twice, three, four or five times at the aryl group;
R₄ and R₅ are independently from each other selected from the group of H, N, C₁-C₄ alkyl group, and (CH₂)ₙ—CR₆R₇R₈;
wherein R₆ and R₇ are independently from each other selected from H or a C₁-C₄ alkyl group, and
R₈ is H, C₁-C₄ alkyl group or a cyclopropyl group;
n is 0 or 1;
or salts, solvates or hydrates thereof.

Preferably, X₂ is at least in para-position. In an embodiment, X₂ is present at the para-position only.

The molecules and compounds according to the present invention targets the new drug target, E1, thus, representing an alternative to the known NS3, NS5a or NS5b inhibitors of hepaci virus, including hepatitis C. In particular, the compounds according to the present invention are new compounds having superior activity for inhibiting virus membrane fusion compared to known compounds including flunarizine or chlorcyclizine.

These compounds are particularly relevant and useful for treating subgroups of hepaci virus infected individuals, namely, individuals having an interfacial hydrophobicity of E1 of Zero or above using the Wimley and White interfacial hydrophobicity scale (White SH & Wimley WC (1999). Annu. Rev. Biophys. Biomol. Struc. 28:319-365). The compounds according to the present invention demonstrates a high-antiviral activity in a whole lifecycle compounds screening assay. In particular, further substituent at the para position to the allyl element modulate and increase the antiviral activity. Particularly, differently substituted amino substituent including a free amino group, triazoles demonstrate a high activity while changes at other positions, e. g. an additional methyl substituent at the alkene is detrimental to the activity. That is, the compounds according to the present invention being substituted at the para position of the phenyl group adjacent to the allyl element (a cinnamyl group) has beneficial effects on antiviral activity. This is also true for functional groups like an acid group.

The term “alkyl” refers to a saturated aliphatic hydrocarbon including straight chain and branched chain groups. The alkyl groups may be substituted or unsubstituted. Substitutions may also be themselves substituted. When substituted, the substituent group is preferably, but not limited to, C1-C3 alkyl, amino, cyano, halogen, C1-C3 alkoxy or hydroxyl.

As used herein, the term C₁ to C₄ alkyl group include a C₁ alkyl group, namely, methyl, an ethyl group (C₂ alkyl group), a propyl group including isopropyl or cylcopropyl, and butyl group. A C₁ to C₄ alkyl group include straight or branched alkyl group, if possible.

In addition, the term halogen includes F, Cl, Br and I.

“Alkoxy” refers to the group —O—R with R being C1-C4 alkyl.

The term C1 to C4 alkoxy group refers to a methoxy, ethoxy, propoxy or butoxy group.

The term “salts” refers to any ionic form of a compound and one or more counter-ionic species (cations and/or anions). The term “salt” additionally includes zwitterionic compounds (i.e. a molecule containing one or more cationic and anionic species, e.g. zwitterionic amino acids). Counter ions present in a salt can include any cationic, anionic, or zwitterionic species. Examples of anions include, but are not limited to: chloride, bromide, iodide, nitrate, sulfate, bisulfate, sulfite, bisulfate, phosphate, acid phosphate, perchlorate, chlorate, chlorite, hypochlorite, periodate, iodate, iodite, hypoiodite, carbonate, bicarbonate, isonicotinate, acetate, trichloroacetate, trifluoroacetate, lactate, salicylate, citrate, tartrate, pantothenate, bitartrate, ascorbate, succinate, maleate, gentisinate, fumarate, gluconate, glucaronate, saccharate, formate, benzoate, glutamate, methanesulfonate, trifluormethansulfonate, ethanesulfonate, benzenesulfonate, p-toluenesulfonate, p-trifluoromethylbenzenesulfonate, hydroxide, aluminates and borates. Examples of cations include, but are not limited to: monovalent alkali, metal cations, such as lithium, sodium, potassium, and cesium, and divalent alkaline earth metals, such as beryllium, magnesium, calcium, strontium, and barium. Also covered by this term are transition metal cations, such as gold, silver, copper and zinc, as well as non-metal cations, such as ammonium salts.

As used herein, the term “pharmaceutically acceptable” is used to refer to those compounds, materials, compositions, and/or dosage forms which are suitable for use in contact with the tissues of human beings and animals without excessive toxicity, irritation, allergic response, or other problem or complication, commensurate with a reasonable benefit/risk ratio.

A “therapeutically effective dosage” of a compound is the amount that is required to inhibit hepaci virus, like HCV entry into cells in order to reduce the hepaci virus, like HCV infection rate to about 10%. In other words, a “therapeutically effective dosage” of the compound is the amount needed to cause an effect in vivo that about 90% of infection will be reduced. It is known in the art that the therapeutically effective dosage of a drug depends on the route of administration.

The present invention also includes pharmaceutically acceptable salts of the compounds according to the present invention, e.g. being present in a pharmaceutical composition according to the present invention. As used herein “pharmaceutically acceptable salts” refers to derivatives of the compounds according to the present invention wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids, and the like. Generally, such salts can be prepared by reacting the free acid or base forms of the compounds with a stoichiometric amount of the appropriate base or acid in water or in an organic solvent, or in a mixture of the two; generally, non-aqueous media like ether, ethyl acetate, alcohols (e.g. methanol), ethanol, iso-propanol, or butanol) or acetonitrile are preferred. The compounds according to the present invention including salts, solvates or hydrates thereof can be prepared using known organic synthesis techniques and can be synthesized according to any of numerous possible synthetic routes.

In an embodiment of the present invention, the compound of general formula I is a compound wherein residue X₁ is N. That is, in an embodiment of the present invention, the compound of general formula I is a piperazine based compound. Alternatively, in case when X₁ is C, the compound according to the present invention is a piperidine based compound.

In another embodiment of the present invention, the halogen of R₁ and R₂ is F. In an embodiment of the present invention, at least one of R₁ and R₂ is F, in another embodiment, both of R₁ and R₂ are F. Other embodiments include the presence of at least two R₁ residues and/or two R₂ residues at the phenyl groups. However, in an embodiment, the halogen, in particular, F, representing R₁ and/or R₂ is in para position.

In another embodiment, the compound of general formula I according to the present invention is a compound wherein X₂ is a C₁ to C₄ alkoxy group, in particular, a methoxy group. Alternatively, the compound of general formula I is a compound wherein X₂ is a NR₄R₅ group wherein at least one of R₄ and R₅ is hydrogen. Further, X₂ is N₃.

Another embodiment identifies a compound of general formula I wherein NR₄R₅ is a group selected from NH₂, NH—CH(CH₃)₂, NH—C-tertbutyl, NH-ethylcyclopropyl. Alternatively, X₂ is N₃. Other embodiments described herein includes compounds of general formula I wherein X₂ is any one of the following structures

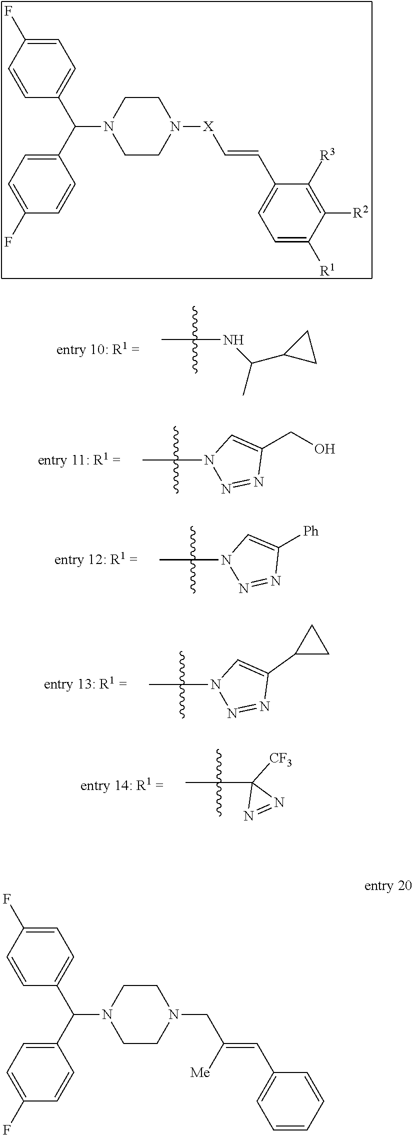

In the context of the present invention, the term “comprising”, “comprises”, “containing” or “contains” include the embodiments of “consisting of” or “consist”.

In an embodiment of the present invention the compound of general Formula I is a compound of the following

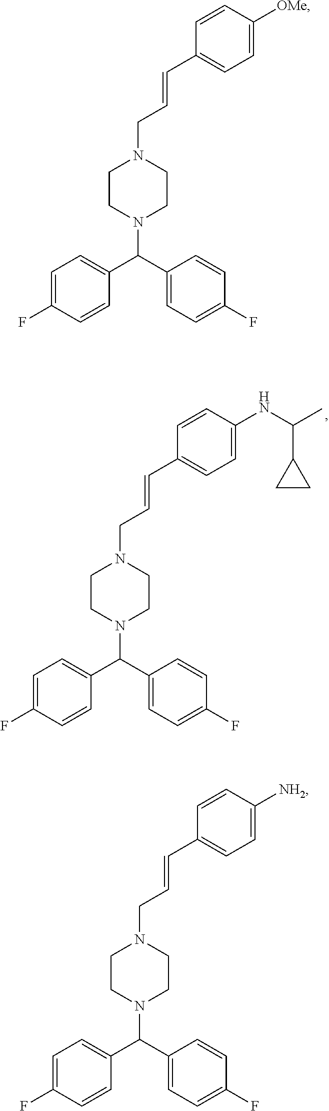

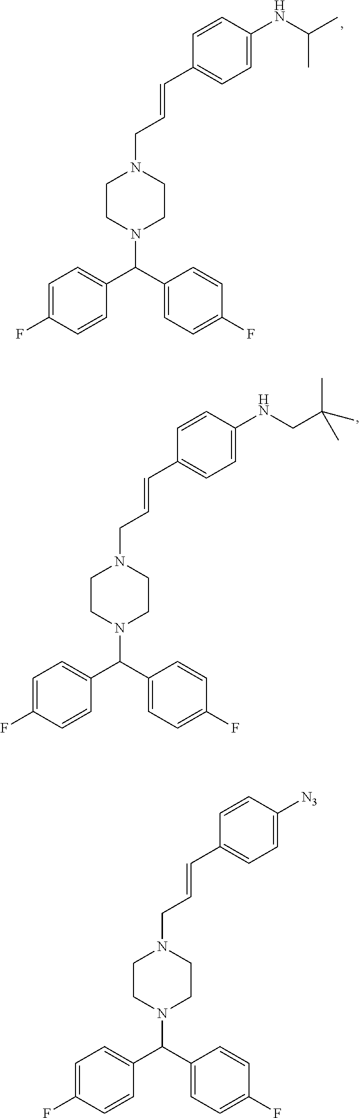

In another embodiment, the present invention relates to a pharmaceutical composition containing a compound of general Formula I according to the present invention. The pharmaceutical composition according to the present invention as well as the compound of general Formula I according to the present invention are useful for preventing and/or treating hepaci virus infection. Hepaci virus infection include non-primate hepaci virus including hepaci virus A, B, D to N of non-primate hepaci virus from horse, rat, gorilla, rodent, bat, or bovine. In an embodiment, the hepaci virus is Hepatitis C virus (HCV).

Compounds according to the present invention may be used in form of its pure compounds or of its salts thereof or in form of solvates, like hydrates. For example, the compounds according to the present invention may be administered in form a pharmaceutical acceptable salts thereof. The pharmaceutical composition according to the present invention may comprise further suitable diluents, excipients, or carriers. The pharmaceutical composition may be administered with a physiologically acceptable carrier to an individual. In a specific embodiment, the term “pharmaceutically accepted” means approved by regulatory agency or other generally recognized pharmacopoea for use in animals and, more particularly, in humans. The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly, for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skin milk, glycerol, propylene glycol, water, ethanol and the like. The pharmaceutical composition, if desired, can also contain minor amounts of wetting or emulsifying agent, or pH buffering agents. These compositions can take the form of solutions, suspensions, emulsion, tablets, pills, capsules, powders, sustained-release formulations, patches and the like. The compositions will contain a therapeutically effective amount of the compounds according to the present invention, preferably in purified form, together with a suitable amount of carrier so as to provide the form for proper administration to the individual. The formulation should suit the mode of administration. The route of administration of the compounds of the present invention depends on the formulation in use. The composition is formulated in accordance with routine procedures as the pharmaceutical composition adapted for intravenous or oral administration to individuals including human beings. Typically, composition for intravenous administrations or solutions in sterile isotonic aqueous buffer.

The term “administered” means administration of therapeutically effective dose or dosage of the pharmaceutical composition or the compounds according to the present invention. The methods are applicable to both human therapy and veterinary applications. The administration of the pharmaceutical composition can be done in a physiologically acceptable carrier as discussed above, including, but not limiting to, orally, subcutaneously, intravenously, intra-arterially, intraorally, intramedullary, intrathecally, intraventiculary, intranasally, intrabronchially, transdermally, intrarectally, intraperitoneally, intramuscularly, intrapulmonarily, vaginally, rectally, or intraoculary. Effective doses may be extrapolated from dose-response curves derived from in vitro animal model tests. As it is known in the art and described herein, adjustments for systemic versus locally delivery, age, body weight, generally health, sex, diet, time of administration, drug interaction and the severity of the condition may be necessary, and will be ascertainable with routine experimentation by those skilled in the art.

In an embodiment of the present invention, the compounds according to the present invention are the only pharmaceutical active agent comprised in said pharmaceutical composition. Moreover, in another embodiment of the present invention, the pharmaceutical composition according to the present invention may contain at least one further anti-hepaci virus inhibitor, like an anti-Hepatitis C virus inhibitor. The at least one further anti-hepaci virus inhibitor, like an anti-Hepatitis C virus inhibitor may be a DAA, like an inhibitor of the NS3 protease, the NSSA protein or the NSSB polymerase. Further, ribavirin may be a further active agent. In particular, the further anti-hepaci virus inhibitor, like anti-Hepatitis C virus inhibitor is at least one of sofosbuvir, dasabuvir, simeprevir (TMC435,)paritaprevir, grazoprevir (MK-5172), daclatasvir, ledipasvir, ombitasvir, velpatasvir, elbasvir (MK-8742). In case where more than one active agent is present in the pharmaceutical composition, the pharmaceutical composition may be adapted for allowing simultaneous, separate or sequential administration of the different active agents.

In addition, the present invention provides a method for the treatment or for the prophylaxis of hepaci virus infection, like Hepatitis C virus infection. The method comprises the step of administering a therapeutically effective amount of a compound according to the present invention. The skilled person is well aware of determining the effective amount, however, for the purpose of the present invention, a therapeutically effective dosage of the compound according to the present invention may preferably from about 1 to 2000 mg/d, preferably from about 10 to 1000 mg/d and further preferably from about 20 to 100 mg/d, which may be administered in one or multiple doses.

The administration in the method for the treatment or prophylaxis according to the present invention may be effected by any route of administration including oral, parenteral, such as subcutaneous intravenous, intramascular, intraperitoral, intrahecal, transdermal, transmucosal, subdural, nasal, local or topical via iontophoresis, sublingual, by inhalation spray, aerosol or rectally and the like in dosage in its formulations optionally comprising conventional pharmaceutical accepted excipients, diluents or carriers.

In an embodiment of the present invention, the compound according to the present invention or the pharmaceutical composition according to the present invention is for use in preventing or treating Hepatitis C virus, in particular, wherein the HCV is HCV of genotype 2. In particular, the compounds of general Formula I or the pharmaceutical composition according to the present invention are for use in treating or preventing hepaci virus infection, like preventing or treating Hepatitis C virus infection, in particular, wherein the HCV is HCV GT2 in individuals being resistant to therapeutic drugs against other targets, including NS3 protease, NSSA phosphoprotein or NSSB polymerase.

Namely, the compounds according to the present invention representing DAAs having the advantage of not being affected by drug resistance against the known DAA.

In another embodiment, the present invention relates to a method for determining effectiveness of prophylactic or therapeutic treatment of hepaci virus infection, like HCV infection, in particular, HCV type 2 infection, comprising:
Determining the interfacial hydrophobicity of the E1 of the hepaci virus, like the E1 of the HCV strain, using the Wimley and White interfacial hydrophobicity scale;
Determining the sensitivity to a diphenylpiperazine or diphenylpiperidine based hepaci virus inhibitor, as well as to the phenothiazine and cycloheptenepiperidine based hepaci virus inhibitors, in particular, a compound of any one of claims 1 to 7,
   wherein a disrupted central hydrophobic region of the E1 protein or a hydrophobicity of the E1 protein lower than zero at the next downstream His residue as has been determined with the Wimley-White hydropathy plot is indicative for reduced sensitivity to a diphenylpiperazine or diphenylpiperidine based hepaci virus inhibitor as well as phenothiazine and cycloheptenepiperidine based hepaci virus inhibitors.

Lower than zero refers to the next His residue downstream and most proximal. That is, the level of predicted hydrophobicity of the most proximal downstream His and continuity of the central hydrophobic region predicts flunarizine sensitivity. As shown in the examples, HCV strains showing a continuous central hydrophobic region and also a hydrophobicity greater than 0 around the proximal His are sensitive to flunarizine. In contrast, strains with a disrupted central hydrophobic region and also hydrophobicity lower than 0 around the proximal His residue are resistant to flunarizine.

A further embodiment identifies a method for determining the therapy regimen of an individual afflicted with hepaci virus infection or being at risk of being afflicted with hepaci virus infection, in particular, having or being at risk of HCV infection, comprising the step of
Providing a sample obtained from said individual and determining the interfacial hydrophobicity of the E1 protein of the hepaci virus, like the E1 of the HCV or its homolog, in particular, using the Wimley-White hydropathy plot;
Analyzing whether the central hydrophobicity region is disrupted or the hydrophobicity is below zero, in particular, the hydrophobicity is below zero at the next downstream His residue applying the Wimley-White hydrophathy plot; Determining the therapy regimen wherein no inhibitor of the E1 protein of hepaci virus, like of HCV or its homolog, in particular, a diphenylpiperazine or diphenylpiperidine based hepaci virus inhibitor as well as to the phenothiazine and cycloheptenepiperidine based hepaci virus inhibitors is applied when the central hydrophobicity region is disturbed or the hydrophobicity is below zero at the next downstream His residue applying the Wimley-White hydropathy plot.

The present inventors recognized that susceptibility of fusion inhibitors of hepaci virus membrane fusion, in particular, Hepatitis C virus membrane fusion depends on the hydrophobicity of the E1 protein of the hepaci virus, like the HCV, in particular, the HCV genotype 2.

The E1 protein of genotype 2a is exemplified by strain J6 with accession number AF177036. The protein sequence of E1 of J6 is SEQ ID No. 1.

The present inventors recognized that the hydrophobic E1 region of the central region from aa265 to aa312 of SEQ ID No. 1, namely, the hydrophobicity region of the E1 protein including the region of aa290 to aa312 of SEQ ID No. 1 is important for susceptibility to fusion inhibitors. Namely, this central hydrophobic E1 region determines the pH range of Hepatitis C virus membrane fusion.

As used herein, the Wimley-White prediction of hydrophobicity within the central region refers to the region from amino acids 265 to 312.

It is recognized by the present inventors that mutations at positions 290, 299, 301 and 310 of SEQ ID No. 1 disturb or disrupt the hydrophobicity in said region. In particular, it has been recognized that mutations M290A, W299N, V301T and P310Q are predictive for disruption of central hydrophobicity as it is the case for HCV strains of genotype 2B.

Of course, other algorithms may be applied for determining hydrophobicity. The skilled person is well aware of suitable algorithms and programs.

The method according to the present invention allows identifying suitable new fusion inhibitors for treating and preventing infection with hepaci virus, in particular HCV. This is particularly true for infection with HCV genotype 2. That is, the surprising identification of relevant changes in the hydrophobicity region result in disrupting the hydrophobicity of the E1 in the central hydrophobicity region allows to search in silico for new inhibitors and lead compounds. Hence, the present invention provides screening methods for identifying suitable inhibitors of membrane fusion of hepaci virus, in particular HCV. In particular, the method according to the present invention allows to predict suitability of a compound as a fusion inhibitor and, thus, as an active agent in preventing or treating infection. This is particularly true for infection with HCV, like HCV GT2.

The present invention will be described further by way of examples without limiting the same:

### Materials and Methods

### HCV Infection Assay

Huh-7.5 cells stably expressing firefly luciferase (Huh-7.5-fluc) were seeded in 12 or 96 well plates. The following day cells were inoculated with infectious viral particles in presence of the test compounds and incubated at 37° C. After 4 hours, the inoculum was aspirated, cells were washed with PBS, fresh medium was applied and cells were incubated at 37° C. Renilla and firefly luciferase activity were quantified 48 hours later to determine infection efficiency and cell viability, respectively.

### Fusion at the Plasma Membrane Assay

The assay were conducted as previously described in Perin, see above. Briefly, Huh-7.5 cells were seeded in 6 well plates (3×10⁵ cell/well) and incubated at 37° C. overnight (18 hours). Subsequently, cells were pre-treated with 5 nM concanamycin A (Con A) for 1 hour at 37° C. (additional steps were carried out in presence of 5 nM Con A to block acidification of endosomes). Cells were inoculated with virus preparations at 4° C. for 2 hours. Subsequently, cells were washed twice with PBS, fresh medium with or without test compounds was added and cells were incubated at 37° C. for one hour. After this, cells were treated with pH 5.0 citric acid buffer for 5 minutes at 37° C. in presence or absence of the test compounds. Cells were washed with PBS again, and fresh medium with or without test compounds was added and cells were incubated at 37° C. for three hours. Finally, cells were washed with PBS, fresh medium without ConA and test compounds was added, and incubation of cells was continued at 37° C. for 48 hours. After this, cells were lysed and Renilla luciferase activity, indicative of infection efficiency, was assessed.

### HCV Whole Life Cycle Compound Screening Assay

Flunarizine derivatives were screened for their antiviral activity in the HCV whole life cycle compound screening assay as previously described in Perin, see above. Briefly, Huh-7.5-fluc cells were transfected with genomic RNA of a GT2a renilla luciferase reporter construct JcR2a Reiss S, et al. Cell host & microbe 2011; 9:32-45. Cells were seeded in 96 well plates and incubated at 37° C. After 4 hours, media containing, serially diluted compounds was added to the cells which were further incubated at 37° C. After 48 hours, media from the transfected cells was harvested and inoculated onto a new set of naïve target Huh-7.5-fluc cells which were incubated at 37° C. for 48 hours. The transfected cells were lysed with water and the cells lysate was analyzed for renilla and firefly luciferase output to assess viral genome replication and cell viability respectively. The target cells were lysed after 48 hours to assess renilla luciferase reporter activity for the whole life cycle.

Plasmids: The full length Jc1 renilla luciferase reporter virus genome JcR2a was described before, Reiss S, et al. Cell host & microbe 2011; 9:32-45. Similarly, the monocistronic renilla luciferase reporter virus genomes H77c/1a/R2a, J4/1b/R2a, JcR2a, J8/2b/R2a, S52/3a/R2a, ED43/4a/R2a, SA13/5a/R2a, HK6a/6a/R2a and QC69/7a/R2a were reported in a previous study, Haid S, et al. Gastroenterology 2012; 143:213-U772. Novel chimeras and point mutants created for this study were cloned into the JcR2a backbone. Specifically, chimeras JcR2a/J8-E1, JcR2a/J8-E1_R1, JcR2a/J8-E1_R2, JcR2a/J8-E1-R3, JcR2a-E1 R4 carrying different portions of J8-derived E1 in the context of the JcR2a genome were generated. Moreover, JcR2a point mutant with four J8-derived residues of E1 subregion R3.1 (S268A, L280V, G283A, A288S) and R3.2 (M290A, W299N, V301T, P310Q) were created. Cloning strategies for these novel constructs can be obtained upon request.
In vitro transcription and electroporation: 10 μg of plasmid DNA was linearized by restriction digestion with an endonuclease Mlul (NEB) and purified using the QiAprep Spin Miniprep kit (QiAgen) according to manufacturer's instructions. The linearized DNA was in vitro transcribed and RNA was purified using NucleoSpin RNA Clean-up kit (Macherey Nagel) according to manufacturer's instructions. Cells counted to a final concentration of 1×107 cells/ml were ressuspended in Cytomix containing 2 mM ATP and 5 mM glutathione and transfected with respective RNA by electroporation. Transfected cells were immediately transferred to complete DMEM before seeding to dishes.
Luciferase infection assay: Huh-7.5-fluc cells seeded one day before in 12 or 96-well plates (5,3×104 cells/well; 1×104/well respectively) were infected with Jct with renilla luciferase for 4 h in presence of different concentrations of compounds. Viruses and compounds were removed, cells were washed with PBS and fresh medium was added. After 48 h, cells were washed with PBS and lysed with MilliQ water. Firefly luciferase activity was evaluated by adding 20 μL of cell lysates into assay buffer (25 mM glycylglycine, 15 mM MgSO4, 4 mM EGTA, 1 mM DTT, 2 mM ATP, 15 mM K2PO4) and luciferin solution (200 μM luciferin, 25 mM glycylglycine, pH 8) and measured for 20 seconds in a luminometer (Centro XS3 LB960; Berthold). Renilla luciferase activity was measured by adding 20 μL of cell lysates into renilla luciferase substrate in PBS (1 μM of coelenterazin; P.J.K.) and measured for 2 seconds in a luminometer (Centro XS3 LB960; Berthold).
Wimley White hydrophobicity analysis: Analysis of E1 protein hydrophobicity was conducted using the Membrane Protein Explorer software package which is made available by the white laboratory (UC, Irvine, USA) under the URL (http://blanco.bio-mol.uci.edu/mpex/). Primary amino acid sequence of full length E1 coding sequences of given strains were used for prediction selecting interfacial hydrophobicity for the analysis. Smoothed hydropathy was plotted, neutralized His residues and predicted hydrophobic segments were include as well.
Statistical Analyses: Data were analyzed using GraphPad Prism version 7.04 or version for Windows (GraphPad Software, La Jolla Calif. USA, www.graphpad.com). IC50 values were calculated using the nonlinear regression model implemented in GraphPad with the least squares fitting method and fixed upper and lower plateaus. Differences in normalized mean infection efficiencies were tested for significance using ordinary two-way ANOVA followed by Dunnett's multiple comparison tests. P-values below the level of 0.05 were considered significant (*p<0.05, **p<0.01, ***p<0.001, ****p<0.0001).
Virtual screening to identify HCV membrane fusion inhibitors: Computational studies were undertaken to identify compounds similar to flunarizine and chlorcyclizine, which exhibit antiviral activity by inhibiting Hepatitis C virus (HCV) cell entry. The structure of N-terminal domain of HCV glycoprotein E1 was obtained from PDB 4UOI. The structure was minimized using forcefield Amber10:EHT of Molecular operating environment (MOE, chemical computing group), Molecular Operating Environment (MOE). Chemical Computing Group Inc., 1010 Sherbooke St. West, Suite #910, Montreal, QC, Canada, H3A 2R7, 2013; 2013.08. Small molecule databases (Approx. 7-8 million small molecules) were screened for those small molecules, which resemble chemically to flunarizine or chlorcyclizine. Resulting small molecules were docked on E1, and were ranked on the basis of binding energy calculations after Molecular dynamics simulations. Once a compound was found active experimentally, further computational calculations involved sub-structure similarity search using fingerprints followed by molecular docking. Details of the methods used are as follows—
Docking: This was a blind docking simulation where entire PDB 4UOI was considered possible binding site. The side chains were kept free to move during force field refinement. Alpha PMI is the placement method used with default settings (sample per conformation=10, maximum poses=250). London dG rescoring was used with Alpha PMI placement. Termination criteria for force field refinement were set as gradient=0.001 and iterations=500.
Molecular dynamics simulations: Top scoring poses from docking were retained for molecular dynamics simulations using amber12 software. We allowed the Leap module of Amber, et al. Journal of computational chemistry 2005; 26:1668-1688, to add missing hydrogen atoms and heavy atoms using the Amber force field (ff10) parameters, Lindorff-Larsen K, et al. Proteins 2010; 78:1950-1958. To neutralize the charge of the system, we added sodium/chloride ions. The model was immersed in a truncated cubical shell of TIP3P water. A time step of 2 fs and a direct-space non-bonded cutoff of 10 A ° were used. After the protein preparation, all systems were minimized to remove the steric clashes that occurred. The systems were then gradually heated to 300 K over a period of 50 ps and then maintained in the isothermal-isobaric ensemble (NPT) at a target temperature of 300 K and a target pressure of 1 bar using a Langevin thermostat and a Berendsen barostat with a collision frequency of 2 ps and a pressure relaxation time of 1 ps, respectively. We constrained hydrogen bonds using the SHAKE algorithm, Ryckaert J-PC, G.; Berendsen, H.J.C. Numerical integration of the cartesian equations of motion of a system with constraints: Molecular dynamics of n-alkanes. J Comput Phys 1977; 23:327-341. We have used the velocity-Verlet algorithm (default algorithm for the Amber MD package) for MD simulations. Particle mesh Ewald (PME) procedure was used to treat long-range electrostatic interactions using default parameters, Darden TY, D.; Pedersen, L. Particle mesh Ewald-an N-log(N) method for Ewald sums in large systems. J Chem Phys 1993; 98:10089-10092. After bringing the systems to our suitable temperature and pressure of 300 K and 1 bar, respectively and equilibrating the system for 500 ps, the production run was continued for 20 ns in the isothermal-isobaric ensemble at the target temperature of 300 K and target pressure of 1 bar using the same Langevin thermostat and Berendsen barostat. The structures in the trajectories were collected at 10 ps intervals. The analysis of trajectories was performed with the Ptraj module of Amber.

### Results

### Flunarizine Resistance Mutations Confer Cross-Resistance to Diphenyl Piperazine, Diphenyl Piperidine, Phenothiazine, Thioxanthene and Cycloheptene Piperidine HCV Entry Inhibitors

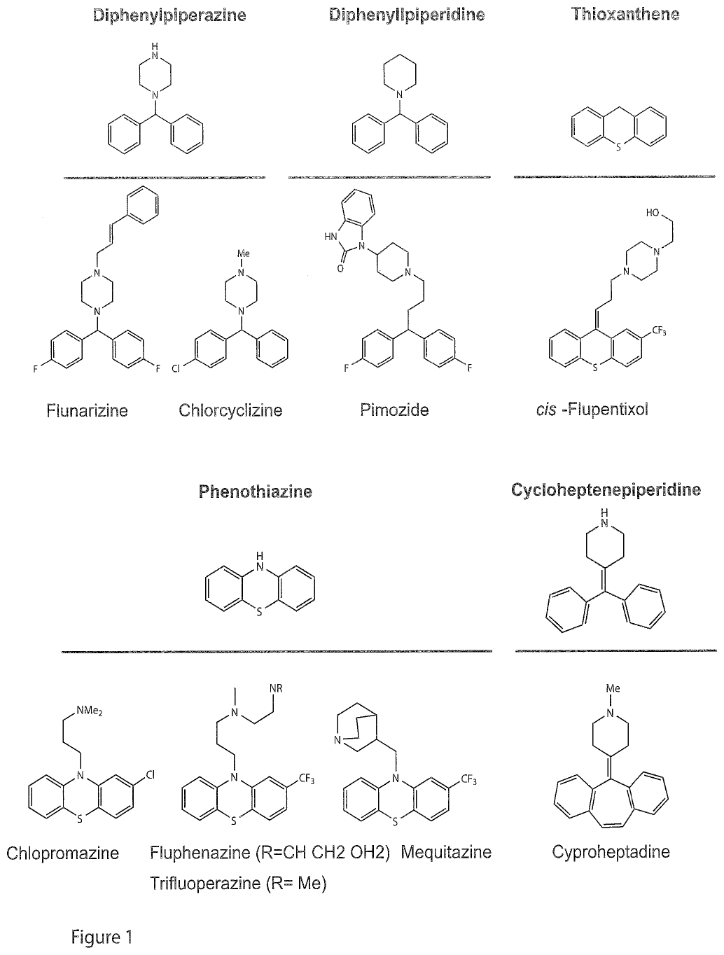

Recently, it has been identified flunarizine, chlorcyclizine (CCZ), pimozide, chlorpromazine, fluphenazine, trifluoperazine, mequitazine, cis-flupentixol and cyproheptadine, which are from the diphenyl piperazine, diephenyl piperidine, phenothiazine, thioxanthene and cycloheptene piperidine families, respectively, as potent HCV entry inhibitors (FIG. 1). Passaging of an HCV genotype 2a virus (Jc1) in the presence of flunarizine yielded a combination of three mutations that map to the E1 and E2 proteins and which confer a ca. 50-fold resistance to flunarizine, Perin see above. These mutations also conferred cross-resitance to pimozide, fluphenazine, and trifluoperazine. To explore if susceptibility to other phenothiazine-, thioxanthene- or cycloheptene piperidine-derived entry inhibitors was also affected by these residues, we infected cells in the presence of increasing doses of these compounds with either the parental Jc1 renilla luciferase virus (JcR2a) or the flunarizine-resistant variant carrying four mutations (M267V and Q289H in E1 and M405T in E2 and I757T in p7; JcR2a-Flun-R which is J6-Flun R) (data not shown). Note that among these four mutations only the three changes in E1 and E2 contribute to flunarizine resistance. Curcumin, a structurally unrelated HCV entry inhibitor, was used as control (FIG. 1). As expected, all compounds dose-dependently inhibited JcR2a infection. Except for curcumin, which equally inhibited JcR2a and the flunarizine resistant Jc1 variant JcR2a-Flun-R, all other compounds were much less active against JcR2a-Flun-R (change in IC50 18 to 275-fold). These results obtained herein indicate that M267V, Q289H and M405T mutations confer cross-resistance to diphenyl piperazine, diphenylpiperidine, phenothiazine, thioxanthene and cyloheptene piperidine based HCV entry inhibitors and suggest that these molecules inhibit HCV cell entry through a common mechanism.

Diphenyl Piperazines, Diphenyl Piperidines, Phenothiazines, and Thioxanthenes Inhibit Low pH-Triggered HCV Cell Membrane Fusion

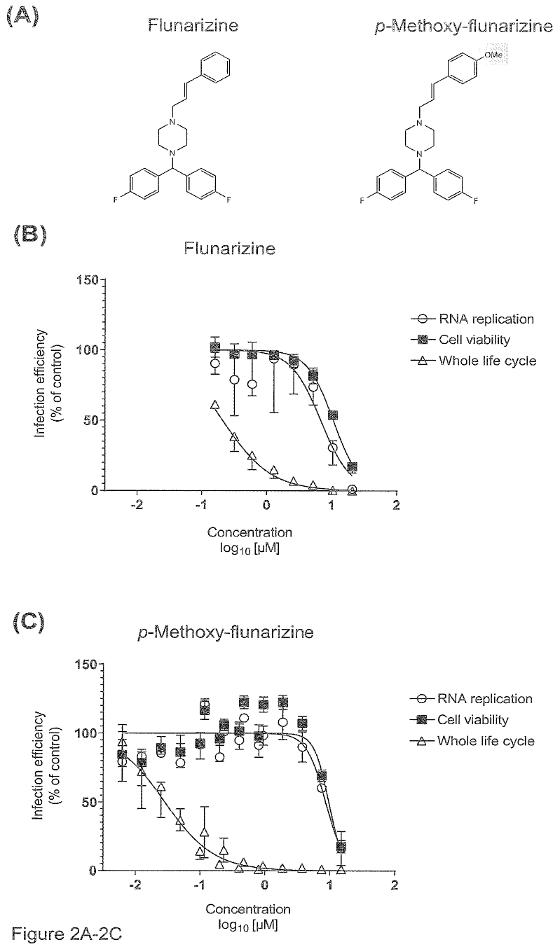

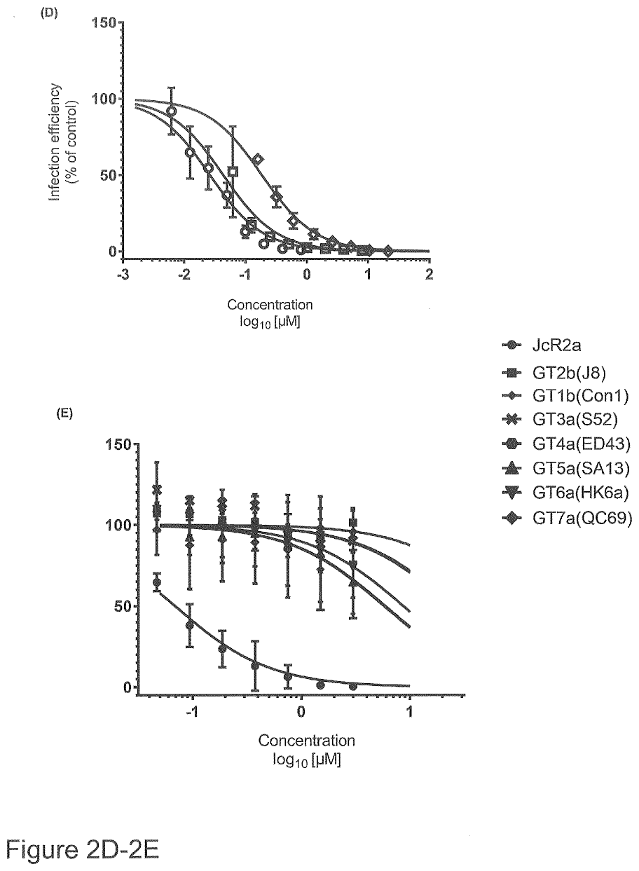

HCV infects liver cells by way of virus-cell membrane fusion, which is essential to deliver the viral genomic RNA into hepatocytes for initiation of infection. Fusion occurs after the virus has interacted with receptors and is internalized into endosomes. These receptor interactions prime the E1/E2 proteins to respond to the acidified pH in endosomes with conformational changes that mediate virus-host cell membrane fusion. When acidification of endosomes is inhibited—for instance by treatment with Concanamycin A that inactivates endosomal ATPases needed for acidification of these organelles—HCV infection is ablated. Under these circumstances infection can be rescued by briefly washing virus exposed cells with a low pH buffer that triggers virus-membrane fusion and allows infection. Using this setup we recently provided evidence that flunarizine specifically inhibits HCV membrane fusion since application of this compound only during the 5 minute low pH wash was sufficient for its full antiviral activity (data not shown). Here we compared the activity of a panel of HCV entry inhibitors in this membrane fusion assays. Compounds tested included a flunarizine-related diphenyl piperazine (chlorcyclizine), a diphenyl piperidine (pimozide), a phenothiazine (mequitazine), and a thioxanthene (cis-flupentixol). In addition we used BJ486K which is flavonoid that is structurally unrelated to flunarizine (FIG. 1) and likely inhibits HCV entry by a different mechanism. In line with this assumption, and in contrast to flunarizine, BJ486K did not inhibit HCV infection when administered only during the low pH washing step (FIG. 2). In contrast, all tested flunarizine-related compounds from the family of diphenyl piperazines, diphenyl piperidines, phenothiazines, and thioxanthenes inhibited HCV infection when added only during the low pH wash. This finding supported the conclusion that these related compounds selectively inhibit low pH-triggered HCV cell membrane fusion.

### Diphenyl Piperidines Preferentially Inhibit of HCV GT2 Membrane Fusion

Among all tested compounds, flunarizine and chlorcyclizine emerged as most potent HCV membrane fusion inhibitors. Therefore, we focused our analysis on these two molecules. Previously we observed that flunarizine preferentially inhibits HCV GT2 viruses including viral strains from subtypes 2a, 2d, 2e, 2k, 2m and 2q and that it was less active against other viral genotypes, see Perin. To explore if cholorcyclizine exhibits a similar preference for GT2 over other HCV genotypes we tested inhibition of HCV strains Con1 (GT1b), J8 (GT2b), S52 (GT3a), ED43 (GT4a), SA13 (GT5a), HK6a (GT6a) and QC69 (GT7a). In contrast to curcumin which was similarly active against all tested viruses, both flunarzine and cholorcyclizine preferentially inhibited Jc1, a G2a virus chimera, and they were less active against a GT2b strain (J8) and all other strains from non-GT2 genotypes tested. Thus, both flunarizine and chlorcyclizine exhibit a similar strain-dependent antiviral activity suggesting that they inhibit HCV membrane fusion through a common mechanism, possibly involving a similar viral target site.

Structure-Activity-Relationship (SAR)-Studies with New Flunarizine Derivatives

We synthesized a series of novel flunarizine derivatives (Table 1). The design of this compound library focused on structural changes in the allylic element and the adjacent arene substituent. Therefore no structural changes in the piperazine ring and the bis(4-fluorophenyl)methane unit. All derivatives were prepared from 4,4′-difluorbenzophenone according to the synthetic protocol reported in reference, Weiwer M, et al. Bioorg Med Chem Lett 2012; 22:1822-1826. With this compound library in hand we tested for their antiviral activity in a whole life cycle compound screening assay as described in Perin, et al. showing that thirty exhibited antiviral activity against the Jc1 reporter virus (Table 1). An additional methyl substituent at the alkene is also not beneficial (entry 20). Several additional substituents at the phenyl ring, particularly in the para position to the allyl element modulate and in selective cases increased the antiviral activity. Particularly, differently substituted amino substituents including the free amino group (entries 3, 6. 7 and 10), triazoles (entries 11-13) need to be mentioned. Noteworthy, substituents that can serve in photo affinity labeling studies (entries 4 and 14) only lead to a moderate loss of activity. Methoxy substituents located in the para and meta positions were found to be beneficial (entries 2 and 19). p-Methoxy-flunarizine (entry 2) exhibited an IC₅₀ of 28 nM in the whole life cycle infection assay while maintaining the typical preference for JcR2a (FIG. 2 and table 1). Cell viability of p-methoxy-flunarizine treated cells was reduced by 50% at a dose of 9.6 μM. Thus, p-methoxy-flunarizine emerged as most powerful compound with an IC50 ca. 2-fold lower than chlorcyclizine and ca. 8-fold lower than flunarizine (entry 1) and a therapeutic index (IC50/CC50) of 335 which is improved by 1,8 and 8,8-fold relative to chlorocyclizine and flunarizine, respectively.

### Viral Determinants of Susceptibility to HCV Membrane Fusion Inhibitors Map to Four Residues in E1

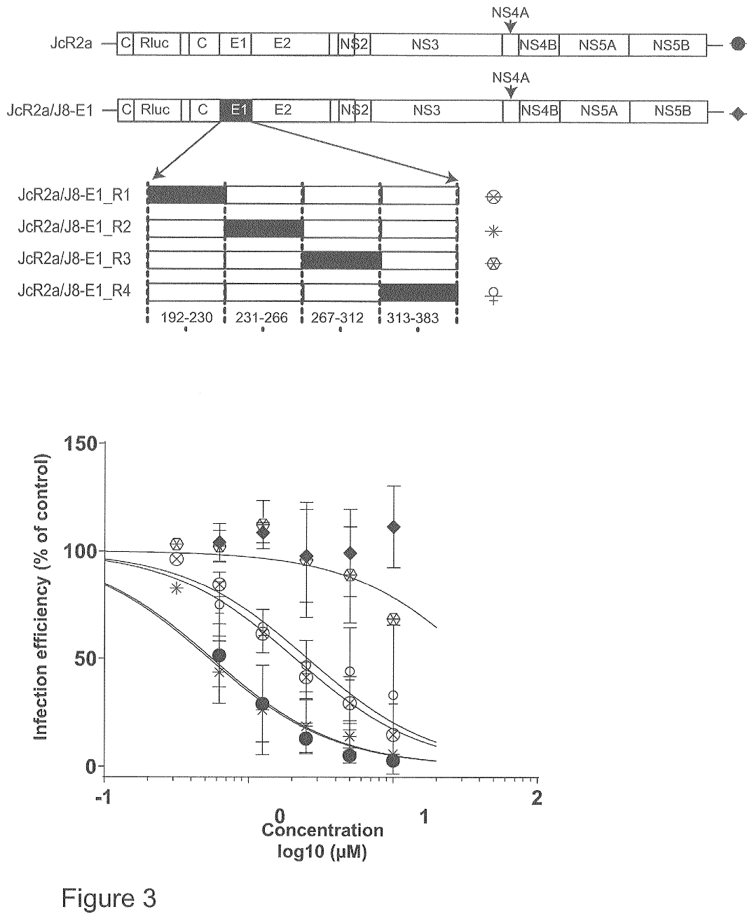

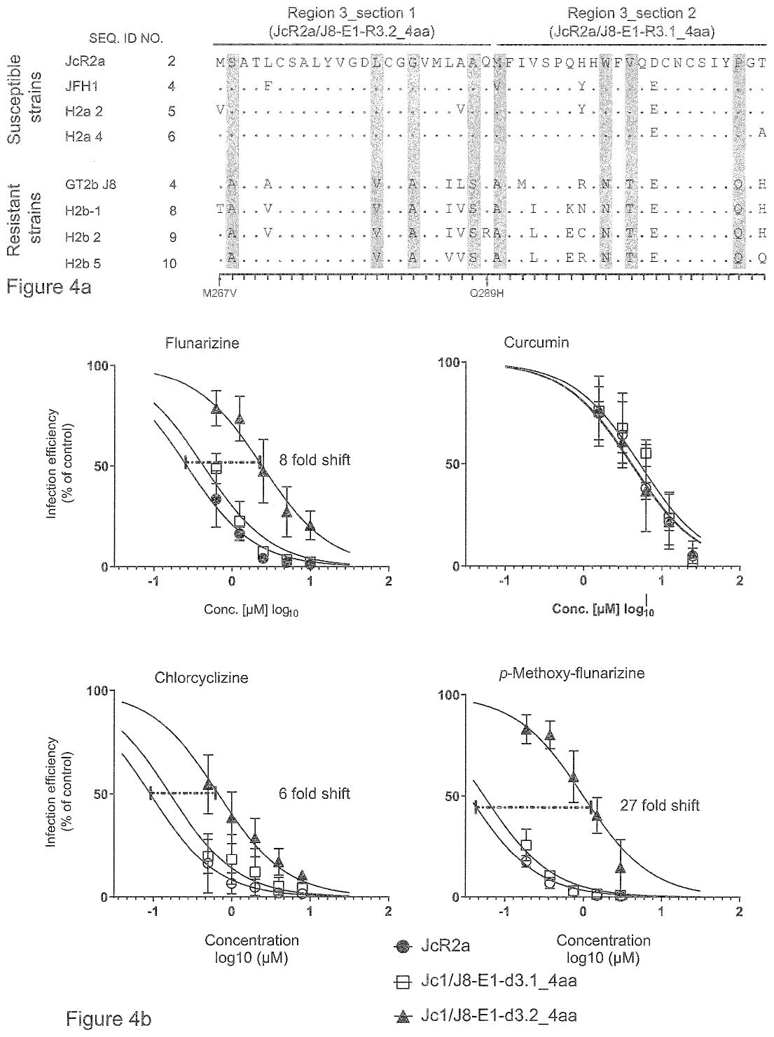

p-Methoxyflunarizine derivative, flunarizine and chlorcyclizine, which emerged as potent representatives of HCV membrane fusion inhibitors, share a preference for inhibiting JcR2a over other HCV strains. While most GT2 strains are susceptible to flunarizine, J8 (GT2b) exhibits pronounced resistance to flunarizine, chlorcyclizine and p-methoxy-flunarizine (FIG. 2). Thus, to map viral determinants responsible for sensitivity to these HCV membrane fusion inhibitors, we took advantage of this difference and created chimeras between the susceptible J6 (GT2a) and the resistant J8 (GT2b) strain. Replacement of Jc1 reporter virus glycoproteins E1 by J8-derived E1 rendered chimeric viruses resistant to flunarizine showing that primarily E1 determines susceptibility to this drug. Of note, replacement of Jct-E1 for J8-E1 did not affect virus RNA replication and release of HCV particles as evidenced by comparable accumulation of luciferase reporter gene activity in lysates of transfected cells and by similar accumulation of released core protein. However, virus infectivity was much decreased suggesting that exchange of E1 selectively impaired cell entry. To compensate for this, virus stocks of the Jc1R2a/J8-E1 chimera were concentrated by ultrafiltration and virus stocks with luciferase transduction efficiency comparable to parental Jc1 were used for the drug resistance testing. Next we created Jc1 chimeras where only specific regions of E1 were replaced by the homologous J8 region. This did not affect RNA replication and virus release but infectivity of the chimeras was partially reduced and was again compensated for by using concentrated virus preparations. While Jc1 chimeras carrying the N-terminal E1 region of J8 (GT2b) (R1; Aa 192-230) or the most C-terminal region (R4; 313-383) displayed partial resistance to flunarizine, a chimera with the internal segments of E1 (R2; 231-266) remained fully susceptible (FIG. 3). In contrast, chimera Jc1/J8-E1_R3 (R3; Aa 267-312) exhibited a resistance profile comparable to Jc1 encoding the flunarizine resistance mutations or to Jc1 encoding the entire E1 protein of J8 (FIG. 3). Notably, two of the three observed flunarizine resistance mutations map to E1 region 3 (M267Y and Q289H; FIGS. 3 and 4A) and this portion of E1 encompasses a putative fusion loop.

To find out which amino acids in this region are critical for conferring susceptibility to HCV membrane fusion inhibitors, we prepared a sequence alignment between four susceptible GT2a strains and four resistant GT2b isolates. We identified eight amino acids which are fully conserved among the sensitive strains and where the resistant strains encode a divergent residue that was fully conserved among the resistant strains. Thus, two additional JcR2a constructs were created encoding either these four N-terminal (i.e. S268A, L280V, G283A, A288S) or these four C-terminal (M290A, W299N, V301T, P310Q) residues of the GT2b strains instead of the cognate GT2a-typical residues. These mutant viruses exhibited RNA replication, virus release and infectivity comparable to parental JcR2a. When we analyzed sensitivity of these virus mutants to entry inhibitors, JcR2a/J8-E1-R3.1_4 aa displayed comparable sensitivity to all tested inhibitors like parental JcR2a. In contrast, JcR2a/J8-E1-R3.2_4 aa was much more resistant to flunarizine, chlorcyclizine, and p-methoxy-flunarizine (8-, 6-, 27-fold, respectively) but not to curcumin. Thus, four polymorphic residues encoded in the E1 region 291 to 312 (M290A, W299N, V301T and P310Q) determine susceptibility of HCV GT2a and 2b isolates to these HCV membrane fusion inhibitors.

Susceptibility of HCV to Membrane Fusion Inhibitors Correlates with their pH Requirements of Fusion Triggering

Many enveloped viruses initiate membrane fusion upon triggering of their fusion proteins in cellular compartments with low pH. Abundant protons in these acidic compartments mediate protonation of viral fusion proteins which in turn triggers conformational changes that bring viral and host membrane into close proximity allowing for injection of viral hydrophobic fusion peptides or fusion loops into the host cell membrane. This process also releases energy that is needed to overcome the energy barrier to virus-cell membrane fusion.

To test if sensitivity of HCV to flunarizine correlates with pH requirements of fusion protein triggering, we used a plasma membrane fusion assay (as described in Perin et al) and washed cell surface-bound HCV particles with buffers ranging from pH5 to pH7. Virus fusion and infection through the natural route of acidified endosomes was prevented by administration of ConA, thus allowing for quantification of virus fusion triggering and infection under defined pH conditions. In line with previously published studies describing the HCV pH requirements, Haid S, et al. JBiolChem 2009, pH 5 efficiently triggered infection of all tested viruses whereas pH 7 did not. However, the tested viruses exhibited significantly different responsiveness to pH values between 5 and 7. Flunarizine sensitive JcR2a exhibited the most stringent pH requirement and efficiently infected cells only when fusion was triggered with a pH of 5.0 (data not shown). In contrast, viruses that exhibited natural resistance to flunarizine (Gt2b, GT3a and GT5a) accepted a significantly broader range of pH including values approaching more neutral conditions. Therefore, natural resistance of HCV to membrane fusion inhibitors correlated with more relaxed requirements of HCV for pH-dependent induction of membrane fusion.

### Resistance Mutations to Membrane Fusion Inhibitors Relax pH Requirement for HCV Fusion

Notably, Jc1 carrying the three resistance mutations to flunarizine also displayed relaxed pH requirements compared to parental Jc1. Moreover, single E1 mutations associated with flunarizine resistance (M267V and Q289H; extended the viral responsiveness towards more neutral pH. This phenotype was not further boosted by combination of both of these changes. Finally, the JcR2a variants encoding the four E1/R3.2_4 aa residues conserved among naturally resistant Gt2b viruses instead of the cognate 2a-typical residues (M290A, W299N, V301T, P310Q) also fused upon triggering with more neutral pH compared to parental JcR2a and compared to the JcR2a variant encoding the four unique GT2b residues of E1/R3.1_4 aa (S268A, L280V, G283A, A288S). Thus, single or combined point mutations that conferred resistance to flunarizine at the same time conferred a more relaxed responsiveness to fusion triggering.

Resistance to Flunarizine, and Relaxed pH Requirements for Fusion Correlate with Modified Hydrophobicity in a Central E1 Region Proximal to the Fusion Loop

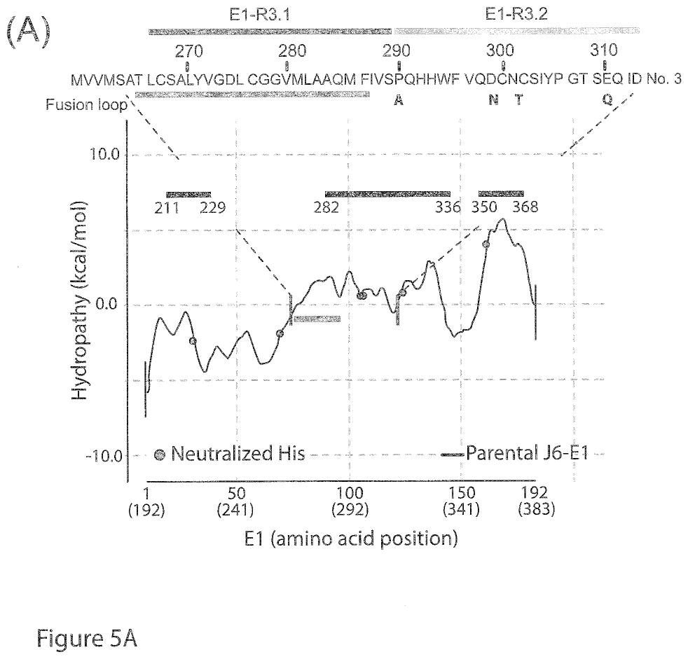

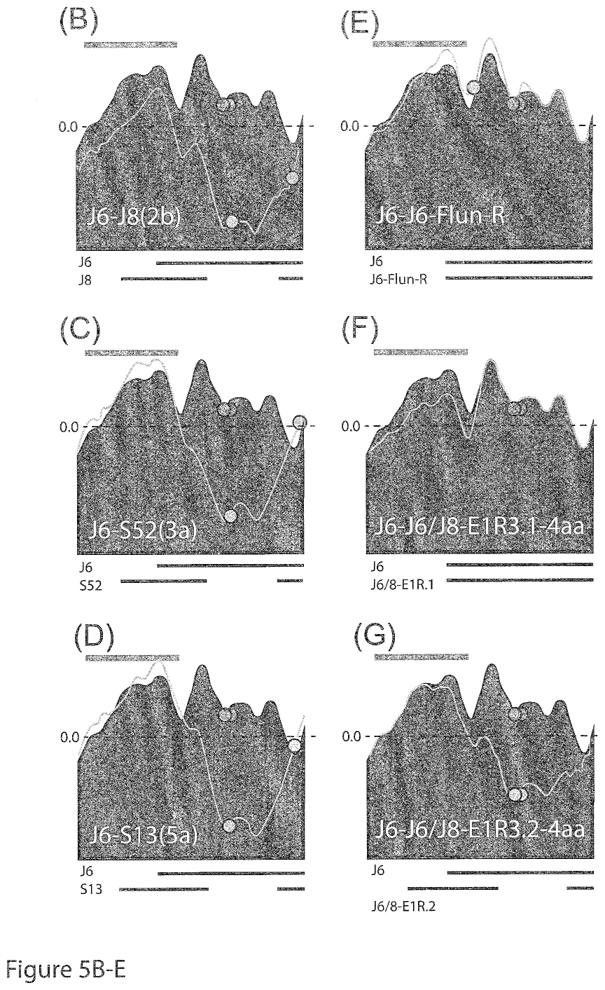

To investigate E1 protein features that may modify sensitivity to membrane fusion inhibitors and pH-dependent membrane fusion, we examined the interfacial hydrophobicitiy of HCV E1 from multiple strains using the Wimley and White interfacial hydrophobicity scale, Wimley WC, White SH. Experimentally determined hydrophobicity scale for proteins at membrane interfaces. Nat Struct Biol 1996; 3:842-848, (FIG. 5). This scale provides a basis for understanding membrane protein folding and membrane insertion. FIG. 5A displays the Wimley-White hydropathy plot for the J6-E1 protein and highlights changes in predicted hydropathy in the central E1 region which encompasses the putative fusion loop and residues controlling sensitivity to flunarizine and pH-dependent membrane fusion. J6 E1 encodes two histidine (His) residues (Aa 297, 298) within a large, continuous hydrophobic region downstream of the predicted fusion loop (FIG. 5A). His has a pKa of ca. 6-7 is the only amino acid whose protonation state changes in a pH range where viral membrane fusion is commonly activated. His is typically uncharged at neutral pH (prediction in FIG. 5A. However, lowering of pH leads to protonation of His, rendering it positively charged. This change can trigger conformational changes by repelling His away from positively charged residues towards negatively charged amino acids where it can engage novel salt bridges. Notably the local environment of His influences its pKa value and in turn the threshold for protonation and membrane fusion. All HCV strains analyzed here (FIG. 5 and Table 2) encode at least one His closely downstream of the putative fusion loop (FIG. 5).

The E1 sequence of the strain J6, AF177036 is shown as Seq. ID. No. 1.

Interestingly, in the flunarizine sensitive J6-E1 which has stringent requirements for pH-triggered fusion, two His in proximity of the putative fusion loop are embedded within a large continuous hydrophobic region. In contrast, in strains with natural resistance to flunarizine and with more relaxed requirement for membrane fusion (e.g. J8 [GT2b], S52 [GT3a], and S13 [GT5a] the His residue proximal to the putative fusion loop is in a much more hydrophilic environment and the continuity of the hydrophobic region is disrupted (FIG. 5A-D). Replacement of four E1-R3.2 residues by amino acids conserved among flunarizine resistant GT2b isolates (J6/J8-E1R3.2-4aa) also disrupts continuity of this hydrophobic region and increases hydrophilicity close to these two His residues (FIG. 5G). Notably, the modified E1 protein (J6/J8-E1R3.2-4aa) not only displays resistance to flunarizine, it also has broadened requirements for low pH-triggered membrane fusion (FIG. 4), thus reinforcing this correlation. In contrast, exchange of the four residues within the E1R.1 region (J6/J8-E1R3.1-4aa) slightly reduces hydrophobicity of the putative fusion loop but does not affect resistance to flunarizine nor alter low pH fusion triggering (FIG. 5F). Collectively, these observations support a model where a central hydrophobic region directly downstream of the putative fusion loop and containing at least one His residue controls pH-dependence of HCV membrane fusion and, correlating with this, sensitivity to HCV membrane fusion inhibitors. The level of predicted hydrophobicity of the most proximal downstream His and continuity of the central hydrophobic region predicts flunarizine sensitivity. All HCV strains showing a continuous central hydrophobic region and also a hydrophobicity greater than 0 around the proximal His are sensitive to flunarizine (Table 2). In contrast, strains with a disrupted central hydrophobic region and also hydrophobicity lower than 0 around the proximal His residue are resistant to flunarizine (Table 2). Thus, for all 11 strains with robust experimental data on flunarizine sensitivity, this simple algorithm stratifies strains into sensitive and in-sensitive strains.

### Discussion

Here we demonstrate the mode of action of several chemically related, HCV entry inhibitors, identified novel derivatives with improved efficacy, defined viral determinants of resistance, mapped the viral target site of these molecules and advanced our understanding how HCV escapes this class of drugs. Moreover, this work also sheds light on E1 protein features that control pH dependent membrane fusion.

Several laboratories reported a broad set of loosely related compounds from the families of diphenylpiperazines, diphenylpiperidines, phenothiazines, thioxanthenes, and cycloheptenepiperidines as potent HCV entry inhibitors. Here we unite and extend these reports by showing that representatives of these chemical classes share a common mode of action which is specific inhibition of HCV membrane fusion. This conclusion is based on several observations: First, an HCV variant with resistance mutations to flunarizine exhibited pronounced cross-resistance to all tested representatives of these chemical scaffolds. In contrast these mutations did not confer resistance to a previously published entry inhibitor with grossly different scaffold (i.e. curcumin). Second, all tested representatives of these compound families were uniquely able to fully inhibit HCV infection when administered only during low pH triggering of the HCV fusion process. In contrast, BJ486K, an HCV entry inhibitor with different chemical properties and alternative mode of action only exerted antiviral activity when present throughout the viral entry process. Finally, flunarizine, p-methoxy-flunarizine, and chlorcyclizine, the most potent among these related molecules and also the related compounds pimozide, trifluoperazine and fluphenazine share a pronounced preference for inhibition of GT2-derived viral strains. These findings suggest that most GT2 strains are particularly vulnerable to this novel class of HCV membrane fusion inhibitors. The specific sensitivity of GT2 viruses to these compounds seems to be linked with a uniquely tight regulation of pH-dependent membrane fusion and/or specific sequence properties of their E1 proteins.

Based on these observations we completed a structure-activity relationship analysis and identified p-methoxy-flunarizine, as most potent compound of this series of inhibitors. p-Methoxy-flunarizine exhibited an IC₅₀ of 28 nM, which is 8-fold better than flunarizine and ca. 2-fold improved compared to chlorcyclizine. Notably, this modification does not alter cross-genotype coverage of the antiviral activity. This is in line with our previous observation that flunarizine-related molecules with modifications in the tricyclic aromatic phenothiazine backbone exhibited somewhat improved cross-genotype antiviral activity (fluphenazine and trifluoperazine. This broad analysis of related compounds permits a more precise definition of the chemical space of these membrane fusion inhibitors. Our analysis particularly demonstrates the importance of the cinnamyl group as attractive element for expanding the SAR-studies for achieving improved antiviral properties. The possibility of synthesizing azido- and 3-(trifluoromethyl)-3H-diazirine derivatives (table 1, entries 4 and 14) paves the way for initiating studies on target elucidation for this group of potential antiviral agents.

Identification of viral determinants for susceptibility to these compounds is important to select patients that may benefit from treatments involving such molecules. This information is also critical to guide development of improved membrane fusion inhibitors with regard to potency and possibly also cross-genotype coverage. By using a series of chimeras between the sensitive J6 strain and a resistant GT2b isolate J8 we pinpointed viral determinants of flunarizine sensitivity. We identified a central hydrophobic region in J6-E1 (J6-E1-R3.2; Aa 290-312) to be most important for controlling sensitivity to this compound. In fact, four residues within this region and which are conserved among sensitive GT2a strains (M/V290, W299, V301, P310) and where resistant GT2b strains encode a conserved alternative reside (A290, N299, T301, Q310) cause and 8-fold, 6-fold or 27-fold shift of the IC50 of flunarizine, chlorcyclizine and p-methoxy-flunarizine, respectively. Notably, these amino acids also influence responsiveness of J6 E1 to low pH-triggered membrane fusion. While parental J6 is sensitive to fluanrizine and fuses only in presence of very low pH, J6/J8-E1R3.2_4 aa is much more resistant and it accepts a more neutral pH for membrane fusion (FIG. 5). These changes are accompanied by a remarkable alteration of hydrophobicity in the central hydrophobic region directly downstream of the putative fusion loop. In the mutant J6 protein (J6/J8-E1R3.2_4aa) the hydrophobic stretch is disrupted and the two proximal His residues end up in a more hydrophilic environment. Notably, HCV strains that have natural resistance to flunarizine also display a disrupted central area of hydrophobicity and their His residue most closely downstream of the putative fusion loop is in a more hydrophilic environment. Taken together these findings suggest that analysis of the hydrophobicity within the E1 region proximal to the putative fusion loop including the assessment of the hydrophobicity around the adjacent His predicts viral sensitivity to flunarizine and related membrane fusion inhibitors. Beyond this, our data demonstrate that viral determinants in this E1 region control requirements for low pH-dependent membrane fusion, possibly by modifying the accessibility and protonation of His residues close to the putative fusion loop. Therefore, these findings provide an algorithm for selection of HCV patients that may benefit from treatments with membrane fusion inhibitors. They also provide new perspectives for development of further improved inhibitors and the offer new insights into the molecular mechanisms that control HCV membrane fusion.

1. A compound of the general formula I 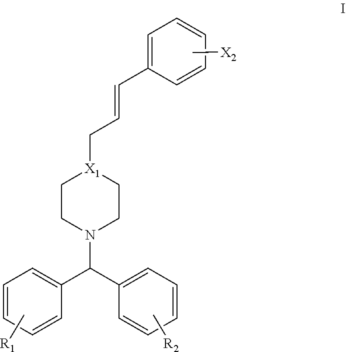
wherein R₁ and R₂ are independently from each other selected from a group of H, Halogen or a C₁-C₄ alkyl group whereby at least one of R₁ and R₂ is a halogen, R₁ and/or R₂ may be present once, twice, three, four or five times at the aryl group,
X₁ is C or N;
X₂ is selected from a OR₃ group or a NR₄R₅ group, or is N₃ wherein
R₃ is H or a C₁-C₄ alkyl group, and X₂ may be present once, twice, three, four or five times at the aryl group;
R₄ and R₅ are independently from each other selected from the group of H, N, C₁-C₄ alkyl group, and (CH₂)ₙ—CR₆R₇R₈;
wherein R₆ and R₇ are independently from each other selected from H or a C₁-C₄ alkyl group, and
R₈ is H, C₁-C₄ alkyl group or a cyclopropyl group;
n is 0 or 1;
or salts, solvates or hydrates thereof.

2. The compound of general formula I according to claim 1 wherein X₁ is N.

3. The compound of general formula I according to claim 1 wherein the halogen of R₁ and R₂ is F.

4. The compound of general formula I according to claim 1 wherein X₂ is a C₁-C₄ alkoxy group.

5. The compound of general formula I according to claim 1 wherein X₂ is a NR₄R₅ group wherein at least one of R₄ and R₅ is H.

6. The compound of general formula I according to claim 1 wherein NR₄R₅ is a group selected from NH₂, NH—CH(CH₃)₂, NH—C-tertbutyl, NH-ethylclyclopropyl, or X₂ is N₃.

7. The compound of general formula I according to claim 1 wherein the compound is selected from the group consisting of

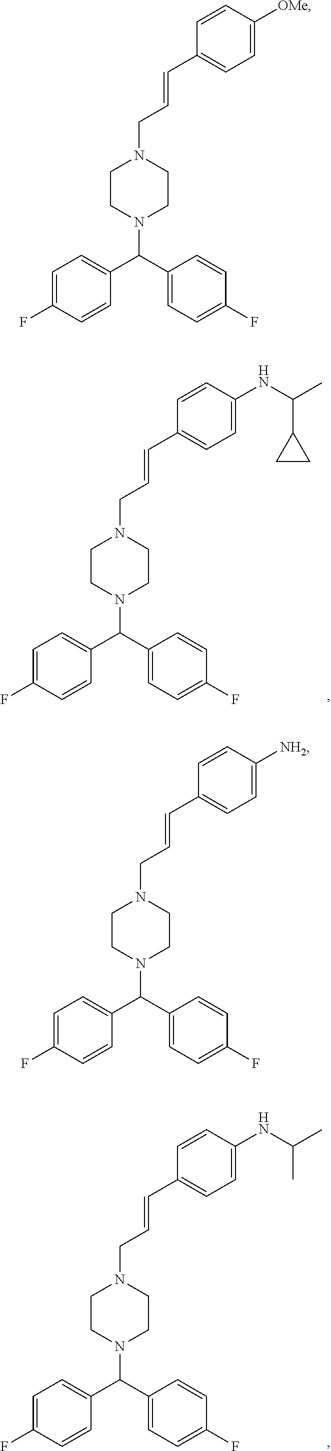

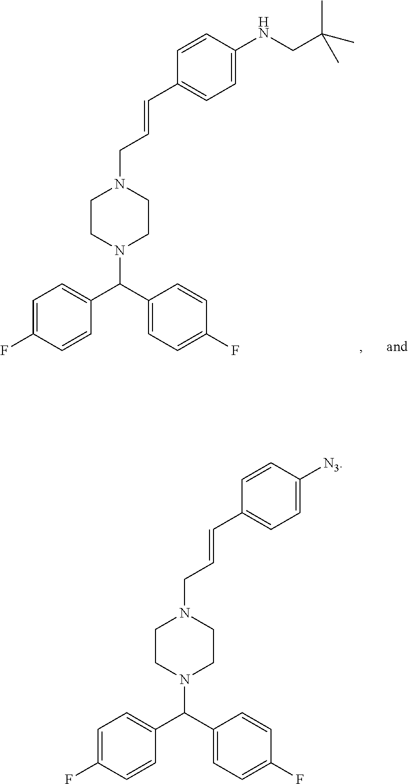

8. A pharmaceutical composition containing a compound of general formula I according to claim 1.

9. A method for preventing or treating hepaci virus infection in a subject in need thereof comprising providing the subject with a compound according to claim 1.

10. The method according to claim 9 wherein the hepacivirus infection is hepatitis C virus infection.

11. The method according to claim 9 further comprising providing the subject with an anti-Hepatitis C virus inhibitor in combination with the compound.

12. A method for determining effectiveness of prophylactic or therapeutic treatment of hepaci virus infection, comprising:
determining an interfacial hydrophobicity of an E1 protein of an hepaci virus using an Wimley and White interfacial hydrophobicity scale;
determining a sensitivity to a diphenylpiperazine or diphenylpiperidine based hepaci virus inhibitor, and to a phenothiazine and cycloheptenepiperidine based hepaci virus inhibitor which is a compound according to claim 1,
wherein a disrupted central hydrophobic region of the E1 protein or a hydrophobicity of the E1 protein lower than zero at a next following His residue as has been determined with the Wimley-White hydropathy plot is indicative for reduced sensitivity to a diphenylpiperazine or diphenylpiperidine based hepaci virus inhibitor as well as to phenothiazine and cycloheptenepiperidine based hepaci virus inhibitors.

13. A method for determining a therapy regimen for an individual afflicted with hepaci virus infection or at risk of being afflicted with hepaci virus infection, comprising the step of
providing a sample obtained from said individual and determining an interfacial hydrophobicity of an E1 protein of an hepaci virus using a Wimley-White hydropathy plot;
analyzing whether a central hydrophobicity region is disrupted or a hydrophobicity is below zero at a next following His residue by applying the Wimley-White hydrophathy plot;
determining a therapy regimen wherein no inhibitor of the E1 protein of the hepaci virus, wherein the inhibitor is selected from the group consisting of a diphenylpiperazine, diphenylpiperidine, phenothiazine, and cycloheptenepiperidine based hepaci virus inhibitors, is applied when the central hydrophobicity region is disturbed or the hydrophobicity is below zero at the next following His residue applying the Wimley-White hydropathy plot.

14. The method according to claim 13 wherein the determining step determines the inhibitor is applied when the central hydrophobic region of E1 is not disturbed or the hydrophobicity is zero or above zero applying the Wimley-White hydrophathy plot.

15. The method according to claim 12 wherein the central hydrophobicity region of the E1 protein comprises a region of aa 290 to aa 312 of SEQ. ID No.1, and further comprising the step of determining whether the HCV strain contain mutations at positions 290, 299, 301 and 310 of SEQ. ID No. 1 which disturb the central hydrophobic region of E1 and the presence of said mutations results in a hydrophobicity below zero by applying the Wimley-White hydropathy plot.

16. The compound of claim 4 wherein the C₁-C₄ alkoxy group is a methoxy group.

17. The method of claim 9 wherein the hepacivirus infection is selected from the group consisting of non-primate hepacivirus like non-primate hepacivirus from horse, rat, gorilla, rodent, bat, bovine, and hepatitis C virus.

18. The method of claim 10 wherein the hepatitis C virus infection is genotype 2.

19. The method of claim 12 wherein the hepaci virus infection is HCV type 2 infection.

20. The method according to claim 13 wherein the central hydrophobicity region of the E1 protein comprises a region of aa 290 to aa 312 of SEQ. ID No.1, and further comprising the step of determining whether the HCV strain contain mutations at positions 290, 299, 301 and 310 of SEQ. ID No. 1 which disturb the central hydrophobic region of E1 and the presence of said mutations results in a hydrophobicity below zero by applying the Wimley-White hydropathy plot.


SEQUENCE LISTING (10 sequences)

SEQ ID NO: 1 - protein, 3033 aa, hepatitis C virus
MSTNPKPQRKTKRNTNRRPQDVKFPGGGQIVGGVYLLPRRGPRLGVRATRKTSERSQPRGRRQPIPKDRRSTGKSWGKPGYPWPLYGNEGLGWAGWLLSPRGSRPSWGPNDPRHRSRNVGKVIDTLTCGFADLMGYIPVVGAPLGGVARALAHGVRVLEDGVNFATGNLPGCSFSIFLLALLSCITTPVSAAEVKNISTGYMVTNDCTNDSITWQLQAAVLHVPGCVPCEKVGNASQCWIPVSPNVAVQRPGALTQGLRTHIDMVVMSATLCSALYVGDLCGGVMLAAQMFIVSPQHHWFVQDCNCSIYPGTITGHRMAWDMMMNWSPTATMILAYAMRVPEVIIDIISGAHWGVMFGLAYFSMQGAWAKVVVILLLAAGVDARTHTVGGSAAQTTGRLTSLFDMGPRQKIQLVNTNGSWHINRTALNCNDSLHTGFIASLFYTHSFNSSGCPERMSACRSIEAFRVGWGALQYEDNVTNPEDMRPYCWHYPPRQCGVVSAKTVCGPVYCFTPSPVVVGTTDRLGAPTYTWGENETDVFLLNSTRPPLGSWFGCTWMNSSGYTKTCGAPPCRTRADFNASTDLLCPTDCFRKHPDTTYLKCGSGPWLTPRCLIDYPYRLWHYPCTVNYTIFKIRMYVGGVEHRLTAACNFTRGDRCNLEDRDRSQLSPLLHSTTEWAILPCSYSDLPALSTGLLHLHQNIVDVQFMYGLSPALTKYIVRWEWVILLFLLLADARVCACLWMLILLGQAEAALEKLVILHAASAASCNGFLYFVIFFVAAWYIKGRVVPLATYSLTGLWSFSLLLLALPQQAYAYDASVHGQIGAALLVMITLFTLTPGYKTLLSRFLWWLCYLLTLGEAMVQEWAPPMQVRGGRDGIIWAVAIFYPGVVFDITKWLLAVLGPAYLLKGALTRVPYFVRAHALLRMCTMARHLAGGRYVQMALLALGRWTGTYIYDHLTPMSDWAASGLRDLAVAVEPIIFSPMEKKVIVWGAETAACGDILHGLPVSARLGREVLLGPADGYTSKGWSLLAPITAYAQQTRGLLGTIVVSMTGRDKTEQAGEIQVLSTVTQSFLGTSISGVLWTVYHGAGNKTLAGSRGPVTQMYSSAEGDLVGWPSPPGTKSLEPCTCGAVDLYLVTRNADVIPARRRGDKRGALLSPRPLSTLKGSSGGPVLCPRGHAVGVFRAAVCSRGVAKSIDFIPVETLDIVTRSPTFSDNSTPPAVPQTYQVGYLHAPTGSGKSTKVPVAYAAQGYKVLVLNPSVAATLGFGAYLSKAHGINPNIRTGVRTVTTGAPITYSTYGKFLADGGCAGGAYDIIICDECHAVDSTTILGIGTVLDQAETAGVRLTVLATATPPGSVTTPHPNIEEVALGQEGEIPFYGRAIPLSYIKGGRHLIFCHSKKKCDELAAALRGMGLNSVAYYRGLDVSVIPTQGDVVVVATDALMTGYTGDFDSVIDCNVAVTQVVDFSLDPTFTITTQIVPQDAVSRSQRRGRTGRGRLGIYRYVSTGERASGMFDSVVLCECYDAGAAWYELTPSETTVRLRAYFNTPGLPVCQDHLEFWEAVFTGLTHIDAHFLSQTKQSGENFAYLTAYQATVCARAKAPPPSWDVMWKCLTRLKPTLVGPTPLLYRLGSVTNEVTLTHPVTKYIATCMQADLEVMTSTWVLAGGVLAAVAAYCLATGCVCIIGRLHINQRAVVAPDKEVLYEAFDEMEECASRAALIEEGQRIAEMLKSKIQGLLQQASKQAQDIQPTVQASWPKVEQFWAKHMWNFISGIQYLAGLSTLPGNPAVASMMAFSAALTSPLSTSTTILLNILGGWLASQIAPPAGATGFVVSGLVGAAVGSIGLGKVLVDILAGYGAGISGALVAFKIMSGEKPSMEDVVNLLPGILSPGALVVGVICAAILRRHVGPGEGAVQWMNRLIAFASRGNHVAPTHYVTESDASQRVTQLLGSLTITSLLRRLHNWITEDCPIPCGGSWLRDVWDWVCTILTDFKNWLTSKLFPKMPGLPFVSCQKGYKGVWAGTGIMTTRCPCGANISGNVRLGSMRITGPKTCMNIWQGTFPINCYTEGQCVPKPAPNFKVAIWRVAASEYAEVTQHGSYHYITGLTTDNLKVPCQLPSPEFFSWVDGVQIHRFAPTPKPFFRDEVSFCVGLNSFVVGSQLPCDPEPDTDVLMSMLTDPSHITAETAARRLARGSPPSEASSSASQLSAPSLRATCTTHGKAYDVDMVDANLFMGGDVTRIESGSKVVVLDSLDPMVEERSDLEPSIPSEYMLPKKRFPPALPAWARPDYNPPLVESWKRPDYQPATVAGCALPPPRKTPTPPPRRRRTVGLSEDSIGDALQQLAIKSFGQPPPSGDSGLSTGAGAADSGSQTPPDELALSETGSISSMPPLEGELGDPDLEPEQVEPQPPPQGGVAAPGSDSGSWSTCSEEDDSVVCCSMSYSWTGALITPCSPEEEKLPINPLSNSLLRYHNKVYCTTTKSASLRAKKVTFDRMQVLDSYYDSVLKDIKLAASKVTARLLTMEEACQLTPPHSARSKYGFGAKEVRSLSGRAVNHIKSVWKDLLEDSETPIPTTIMAKNEVFCVDPTKGGKKAARLIVYPDLGVRVCEKMALYDITQKLPQAVMGASYGFQYSPAQRVEFLLKAWAEKKDPMGFSYDTRCFDSTVTERDIRTEESIYRACSLPEEAHTAIHSLTERLYVGGPMFNSKGQTCGYRRCRASGVLTTSMGNTITCYVKALAACKAAGIIAPTMLVCGDDLVVISESQGTEEDERNLRAFTEAMTRYSAPPGDPPRPEYDLELITSCSSNVSVALGPQGRRRYYLTRDPTTPIARAAWETVRHSPVNSWLGNIIQYAPTIWARMVLMTHFFSILMAQDTLDQNLNFEMYGAVYSVSPLDLPAIIERLHGLDAFSLHTYTPHELTRVASALRKLGAPPLRAWKSRARAVRASLISRGGRAAVCGRYLFNWAVKTKLKLTPLPEARLLDLSSWFTVGAGGGDIYHSVSRARPRLLLFGLLLLFVGVGLFLLPAR

SEQ ID NO: 2 - protein, 46 aa, hepatitis c virus JcR2a
MSATLCSALYVGDLCGGVMLAAQMFIVSPQHHWFVQDCNCSIYPGT

SEQ ID NO: 3 - protein, 49 aa, hepatitis C virus JcR2a J6
MVVMSATLCSALYVGDLCGGVMLAAQMFIVSPQHHWFVQDCNCSIYPGT

SEQ ID NO: 4 - protein, 46 aa, hepatitis C virus JFH1
MSATFCSALYVGDLCGGVMLAAQVFIVSPQYHWFVQECNCSIYPGT

SEQ ID NO: 5 - protein, 46 aa, hepatitis C virus H2a 2
VSATLCSALYVGDLCGGVMLVAQMFIVSPQYHWFVQECNCSIYPGT

SEQ ID NO: 6 - protein, 46 aa, hepatitis C virus H2a 4
MSATLCSALYVGDLCGGVMLAAQMFIVSPQHHWFVQECNCSIYPGA

SEQ ID NO: 7 - protein, 46 aa, hepatitis C virfus GT2b J8
MAATACSALYVGDVCGAVMILSQAFMVSPQRHNFTQECNCSIYQGH

SEQ ID NO: 8 - protein, 46 aa, hepatitis c virus H2b-1
TAATVCSALYVGDVCGAVMIVSQAFIISPKNHNFTQECNCSIYQGH

SEQ ID NO: 9 - protein, 46 aa, hepatitis C virus H2b-2
MAATVCSALYVGDVCGAVMIVSRAFILSPECHNFTQECNCSIYQGH

SEQ ID NO: 10 - protein, 46 aa, hepatitis Cvirus H2b 5
MAATLCSALYVGDVCGAVMVVSQAFMLSPERHNFTQECNCSIYQGQ